(19) United States
(12) Patent Application Publication
(10) Pub. No.: US 2017/0231733 A1
(43) Pub. Date: Aug. 17, 2017

(54) A DENTAL LIGHT DEVICE

(71) Applicant: 3M INNOVATIVE PROPERTIES COMPANY, St. Paul, MN (US)

(72) Inventors: Rudolf Schmid, Eichenau (DE); Heiko Duwensee, Landsberg (DE); Korbinian Schepke-Gerlach, Gauting (DE); Christian A. Richter, Feldafing (DE)

(21) Appl. No.: 15/329,759

(22) PCT Filed: Jul. 28, 2015
(86) PCT No.: PCT/US2015/042342

§ 371 Date: Jan. 27, 2017

(30) Foreign Application Priority Data
Jul. 29, 2014 (EP) 14178867.9

(51) Int. Cl. A61C 13/15; A61N 5/06; A61C 17/022; A61C 1/08; A61C 1/00

(52) U.S. Cl. CPC A61C 19/004; A61C 1/088; A61C 1/0061; A61C 17/022; A61N 5/062; A61N 2005/0606; A61N 2005/063; A61N 2005/0651; A61N 2005/0663; A61N 2005/0666; A61N 2005/067

(57) ABSTRACT

A dental light device which has a light output for providing blue light, and an air outlet arranged adjacent the light output. The air outlet is connected or connectable to an air supply. The dental light device provides for simultaneously irradiating an object and cooling the object.

### FIELD OF THE INVENTION

The invention relates to a dental light device, and in particular to a dental light device which comprises a light output for providing blue light, and an air outlet arranged adjacent the light output.

### BACKGROUND ART

Light-curable or light-hardenable materials are widely used in dentistry for the restoration of teeth, for example for filling a cavity in a tooth. Such materials typically can be made to provide optical characteristics that resemble those of natural teeth, which make those materials a favored alternative to unpleasant looking amalgam materials, for example.

Light-hardenable materials often include a polymerizable matrix material and filler materials including colorants, and may initially be generally soft or flowable so that they can be applied in a desired location and shape. For example, for restoration of a tooth the dental material may be filled into a tooth cavity and shaped so that the restored tooth resembles a natural tooth. Once the desired shape has been formed, the material may be hardened by exposing it to light of a desired wavelength and for a certain material dependent time period. The light typically activates photoinitiators in the dental material that cause the matrix material to polymerize.

The use of dental materials that are hardenable by blue light of a wavelength of between about 450 and 500 nm has become common in dentistry. Accordingly, dental light irradiation devices used for hardening such dental materials typically emit light at such wavelengths and typically are adapted for automatically controlling the light emission for only a pre-selected or pre-selectable time period. Such a dental light irradiation device, for example, is available from 3M ESPE, Germany, under the trade designation Elipar™ S10 LED Curing Light.

A variety of light devices have been developed or proposed. For example U.S. Pat. No. 5,147,204 discloses a light emitting apparatus for curing photocurable dental materials. The apparatus includes a handpiece having a housing, a depending handle and a detachable light guide for guiding light from a lamp in the housing to a front end of the light guide. The light guide is received in a head connected to the housing. The device has a blower mounted in a rear portion of the device for cooling the lamp. In operation, the blower draws air through a series of openings located in the head for circulation around the lamp and discharge through a series of rear apertures formed in the housing.

Normally irradiating a dental material causes that portion of the dental material to harden, which is exposed to sufficiently intense light emitted from the device. Very small amounts of dental material typically can be hardened by activating the device once for the desired preselected operating time period. However for filling larger cavities in a tooth typically the dental material is provided in several portions and hardened successively. Further to harden larger amounts of the dental material the light device must be repositioned one or several times to make sure all relevant portions of the dental material get exposed to light.

Although there are a variety of light devices on the market there is still a desire to provide a device that is relatively convenient in handling and which is useful in hardening even larger amounts of dental material relatively quickly. Further such a device is desirably inexpensive. There is also a desire to provide a device which can be used at a relatively high level of hygiene.

### SUMMARY OF THE INVENTION

The invention relates to a dental light device, particularly for irradiating light-hardenable dental materials for causing such dental materials to harden. The dental light device comprises a light output for providing blue light, and an air outlet arranged adjacent the light output. The air outlet is connected or connectable to an air supply.

For the purpose of the present specification “blue light” refers to light having a wavelength within a range of about 430 nm (nanometers) and about 490 nm and a peak wavelength within a range of about 444 nm and 453 nm. Further such blue light preferably substantially does not comprise light at wavelengths outside the range of about 430 nm and about 490 nm. For example at least 90%, more preferably 95% of the light quantity emitted from the device is formed by blue light having a wavelength within a range of about 430 nm and about 490 nm.

It is preferred that the dental light device operates with air, for example optionally conditioned (for example filtered and/or pre-warmed) ambient air. The present invention encompasses however the operation with any appropriate gas which can be used in a patient's mouth.

The invention is advantageous in that it allows for irradiating a tooth to be restored at a maximized time and/or at a maximized intensity. This is because the dental light device of the invention is adapted for simultaneously irradiating and cooling an object, for example a tooth filled with a dental material.

In an embodiment the light output is adapted for emitting light toward a working area with the air outlet being arranged to guide air supplied through the outlet to the working area or essentially the working area. Accordingly the dental light device may be configured such that a light beam emitted from the light output and an air stream emitted from the air output overlap or cross at a distance from the light output and air outlet outside the dental light device. The dental light device preferably comprises a body portion and a tip portion with each of the body portion and the tip portion having a front end and a rear end. The body front portion and the tip rear portion are preferably connected to each other. Further the tip front portion forms a head that comprises the light output and the air outlet. The air outlet may be formed by one opening or by a plurality of openings. In a preferred embodiment the air output and the light output are spaced by less than 20 mm, measured between a center of the air output and a center of the light output on a straight line between the centers. The center of the light output may be formed by the center of a light source. In case of a plurality of light sources the center may be determined by averaging between the positions of the individual light sources. Further, the center of the air output may correspond to a center of the opening forming the light output.

In one embodiment the head of the tip portion comprises a transparent cover forming the light output and one or more holes in the cover forming the air outlet. In this embodiment the air stream and the light beam are emitted in essentially the same direction. Thus the air is emitted within the light beam. Accordingly a cooling effect of the irradiated object, for example a tooth to be restored can be achieved relatively independent from the distance between the head of the tip portion and the object. Further the magnitude of the air stream velocity automatically chances along with the light intensity dependent from the distance between the head of the tip portion and the object. For example an irradiation of the object from a short distance to the object typically causes the object to be irradiated at relatively high light intensity and the object to be heated at a high heating rate and accordingly also causes the air stream velocity to reach the object at a high velocity and therefore to be cooled at high cooling rates.

In one embodiment the tip portion forms a light guide. The light guide may be formed by a hollow, optionally mirrored structure, or by a solid transparent structure, for example a bundle of glass-fibers. The head optionally comprises a light deflector. The light deflector is preferably arranged for deflecting light that is guided through the light guide in a first direction in a transverse second direction. The light deflector may be formed by a mirror or a prism. Further the light deflector may be integrally formed by the solid transparent structure, for example by a curved end of the solid transparent structure. The light deflector enables the use of the tip portion for irradiating an object in a patient's mouth in relatively narrow spaces.

In a preferred embodiment the dental light device of the invention is of wireless configuration. Although in one embodiment the dental light device may be connected to an air supply, in the preferred embodiment the dental light device comprises the air supply. The dental light device further preferably comprises a battery, in particular a rechargeable or replaceable battery. The battery may allow for powering the dental light device without the need of an external connector cable. Further the dental light device of the invention preferably comprises a light source. The light source may be in the form of a LED (Light Emitting Diode), in particular a single LED (for example a high power LED) or a single laser diode, in particular a single laser diode. The light source is preferably configured to emit blue light.

In a further embodiment the dental light device of the invention comprises a heat sink for dissipating heat generated by the light source. The dental light device further preferably has an air channel extending between an air inlet and the air outlet. The heat sink is optionally at least partially or entirely arranged within the air channel. Thus the air stream may be pre-warmed prior to its emission through the air outlet.

In one embodiment the air supply comprises a motor-drivable fan. The fan is preferably adapted for generating an air stream in a direction from the air inlet toward the air outlet. The fan is preferably arranged within the air channel.

In one embodiment the air channel is formed at least partially by a tube extending between the fan and the front end of the body portion of the dental light device. The tube may extend through at least a part of the body portion to bypass the heat sink. Thus any significant warming of the air stream may be avoided, if desired. Accordingly the air outlet may be connected to the air supply via a passageway in the tip portion between the air outlet and the rear end of the tip portion and the tube which extends between the front end of the body portion and the air supply. The tube and the passageway are preferably sealingly connected for fluid communication in a situation in which the tip portion and the body portion are attached to each other.

In a further embodiment the heat sink is arranged downstream of the fan. Further the dental light device may comprise an (preferably exchangeable) air filter downstream the fan and the heat sink.

In a further embodiment the dental light device of the invention further comprises electronics for controlling the light output at different operating modes and controls for selecting a particular operating mode from the different operating modes. The different operating modes preferably concern different time periods for which the light output remains activated before it deactivates automatically. Accordingly the dental light device may have an automatic switching-off delay after switching on with the time period of the delay being user determinable.

In a further embodiment the dental light device of the invention has means for adjusting a flow rate of air emitted from the air outlet. For example the flow rate may be adjusted by adjusting the speed of the fan. The dental light device may therefore have controls and a corresponding control circuit for adjusting the fan speed. This helps adjusting the air stream to an individual patient's sensation of the cooling, or for adjusting the air stream for use for other purposes, for example for cleaning or trying an area in the patient's mouth. The dental light device may therefore be operable in a mode in which the air stream is activated during the light output is deactivated.

### BRIEF DESCRIPTION OF THE FIGURES

FIG. 1 is a perspective view of a dental light device according to an embodiment of the invention.

### DETAILED DESCRIPTION OF THE INVENTION

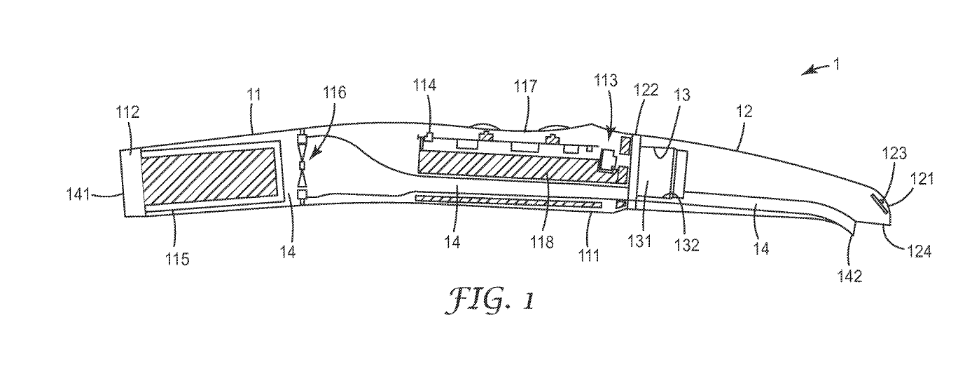

FIG. 1 shows dental light device 1 according to the invention. The device 1 has a body portion 11 and a tip portion 12. The body portion 11 and the tip portion 12 are connected to each other via magnetic coupling 13. The magnetic coupling 13 in the example is formed by a magnetic plug 131 formed at a front end 111 of the body portion 11 and a steel socket 132 formed at a rear end 122 of the tip portion 12. The magnetic plug 131 and the steel socket 132 are of a cylindrical shape. Thus although the magnetic coupling 13 provides for a tight connection between the body portion 11 and the tip portion 12, the magnetic coupling 13 still enables a rotation of the body portion 11 and the tip portion 12 relative to each other.

The body portion 11 in the example includes preferably all electrical components required for operation of the device. Further the tip portion 12 is preferably free of any electric components. Thus the tip portion 12, which normally is the portion used in a patient's mouth, is provided with sufficient robustness to survive multiple disinfections.

In particular the body portion 11 of the device 1 comprises a light source 113, which in the example is a blue laser diode, electronics 114 for operating the light source 113 and a rechargeable battery 115 for powering the device 1. The skilled person will recognize that the electronics 114 may be configured to provide further functions. In particular the electronics 114 may have a charger circuit for controlling the charging of the battery 115 and security circuits for avoiding overheating and/or short circuits etc. The body portion 11 further has controls 117, in particular one or more buttons (not illustrated in detail), for selecting an operating mode of the device 1, for activating the device 1 (for switching on) and for deactivating the device 1 (for switching off). The device 1 of the example has different predetermined operating modes. A select button is provided for pre-selecting the operating mode. In this example at least some of the operating modes relate to a time period over which the light source 113 after activation is maintained activated until it is automatically deactivated. For activation of the light source 113 and for activating the operating mode the device 1 has a start button. The start button may be further configured for deactivating the activated light source at any time. Thus a user may for example pre-select a time period of 10 seconds by using the select button. Pressing the start triggers the light source and the operating mode to be activated. The device 1 in the selected and activated operating mode causes the light source to stay activated and causes the light source to automatically deactivate 10 seconds later. Typical pre-selectable time periods may be for example 5, 10, 15, 20 and 25 seconds.

The device 1 further has an air channel 14 extending between an air outlet 142 at a front end 121 of the tip portion 11 to an air inlet 141, which in this example is arranged at a rear end 112 of the body portion 11. Accordingly the air channel 14 runs between the rear end 112 of the body portion 11 all the way through the device 1 to the front end 121 of the tip portion 11. At the transition between the body portion 11 and the tip portion 12 the air channel preferably comprises a ring-shaped configuration on at least one of the body portion 11 and the tip portion 12, and the ring-shaped configuration is arranged concentric to the coupling 13. Thus a rotation of the body portion 11 and the tip portion 12 relative to each other does not affect the permeability of the air channel 14.

The body portion 11 further has a fan 116 which is arranged and operable to generate an air stream in a direction from the air inlet 141 toward the air outlet 142. The fan 116 is powered by the battery 115 and controlled by the electronics 114. In an alternative embodiment (not shown) the device may have a replaceable or refillable air pressure tank and an electrically operable control valve for controlling the release of air from the tank. In yet another embodiment the device may have an interface for coupling the device with an external air supply, for example as it may be provided at a dentist's chair. The fan 116 is arranged within the air channel 14, downstream the battery 115 and upstream the light source 113. The light source 113 is arranged in thermal contact with a heat sink 118. In one example (indicated by dashed lines) the heat sink 118 may extend into the air channel so that the air stream cools the heat sink and the thereby the air stream is warmed up. This provides an air stream which is warmed up, for example toward the temperature of a human body. Accordingly a treatment using the air stream may be made relatively convenient for the patient.

The tip portion 12 in the example comprises a light deflector 123 which in the example is formed by a reflective mirror. The light deflector 123 is arranged and configured to deflect light emitted from the light source 113 at an angle of approximately 90 degrees toward a light output 124. This is to enable a convenient use of the device even in narrow spaces in the patent's mouth. In another example the light deflector may however be formed by other means, like for example one or more prisms or transparent fibers. In still another example the tip portion 12 may comprise a light guide formed from one or a plurality of transparent fibers, for example glass fibers, which are bent by approximately 90 degrees adjacent the front end 121 of the tip portion 12. Independent from the particular embodiment of the tip portion 12 regarding deflecting and/or guiding light, all of the embodiments comprise the section of the air channel 14 extending through the tip portion. The light output 124 and the air outlet 142 are arranged adjacent and in a manner such that the air stream emitted from the air outlet 142 crosses the light beam emitted from the light output 124 at a determined distance outside the tip portion 12. The area at which the air stream and the light beam intersect preferably corresponds to a working area at which the device may be used for hardening the dental material. The dental material is typically hardened by exposing it to the light that is emitted from the light output. Accordingly some of the light energy typically causes the dental material to warm up over time. In case the dental material is hardened in a cavity in the patient's mouth such warming is acceptable to some extent but nevertheless generally undesired due to a variety of reasons. Firstly a dentist using the device typically does not receive any feedback about the actual temperature of the dental material. For filling larger cavities in a patent's tooth the device may have to be used over a relatively long time period, for example for subsequently hardening several portions of dental material, so that the material may be gradually warmed up over the acceptable temperature. Further any change of temperature, like warming up during hardening and cooling after, may cause discomfort for the patient. By guiding the air stream emitted from the air outlet 142 of the device 1 toward the dental material while it is hardened over-warming can be avoided. Further it has been found that the air stream further can be used to dry and keep dry any surface in the patient's mouth. In particular the light device may be used to dry a cavity in a tooth of a patient prior to inserting the dental material into the cavity. Further any portion of dental material may be hardened and kept dry during hardening so that a further portion of dental material can be applied directly onto the previously hardened portion. This facilitates for example filling of a patent's tooth because the need to change between the light device and a separate air supplying device (as it is for example available at a dentist's chair) may be minimized.

1. A dental light device, comprising; a light output for providing blue light; and an air outlet arranged adjacent the light output, wherein the air outlet is connected or connectable to an air supply.

2. The dental light device of claim 1, wherein the light output is adapted for emitting light toward a working area and wherein the air outlet is arranged to guide air supplied through the outlet to essentially the working area.

3. The dental light device of claim 1, further comprising a body portion and a tip portion, each of the body portion and the tip portion having a front end and a rear end, wherein the body front portion and the tip rear portion are connected to each other, and wherein the tip front portion forms a head that comprises the light output and the air outlet.

4. The dental light device of claim 3, wherein the tip portion forms a light guide with the head optionally comprising a light deflector.

5. The dental light device of claim 1 being of wireless configuration and comprising the air supply.

6. The dental light device of claim 1, further comprising a light source in the form of a single LED or a single laser diode.

7. The dental light device of claim 6, further comprising a heat sink for dissipating heat generated by the light source and an air channel extending between an air inlet and the air outlet, and wherein the heat sink is arranged within the air channel.

8. The dental light device of claim 7, wherein the air supply comprises a motor-drivable fan adapted for generating an air stream in a direction from the air inlet toward the air outlet.

9. The dental light device of claim 8, wherein the heat sink is arranged downstream of the fan.

10. The dental light device of claim 8, further comprising an air filter downstream the fan and the heat sink.

11. The dental light device of claim 1, further comprising a battery, electronics for controlling the light output at different operating modes and controls for selecting a particular operating mode from the different operating modes.

12. The dental light device of claim 11, wherein the different operating modes concern different time periods for which the light output remains activated before it deactivates automatically.

13. The dental light device of claim 1, further comprising a fan that adjusts a flow rate of air emitted from the air outlet.
